(19) United States
(12) Patent Application Publication
(10) Pub. No.: US 2024/0374130 A1
(43) Pub. Date: Nov. 14, 2024

(54) OPERATING ELEMENT FOR OPHTHALMOLOGICAL DEVICES, WITH TARGETED BLOCKING AND/OR ENABLING OF INDIVIDUAL FUNCTIONS

(71) Applicant: Carl Zeiss Meditec AG, Jena (DE)

(72) Inventors: Joerg REINHARDT, Erfurt (DE); Dietrich MARTIN, Jena (DE)

(21) Appl. No.: 18/654,806

(22) Filed: May 3, 2024

(30) Foreign Application Priority Data
May 9, 2023 (DE) 10 2023 204 251.7

(51) Int. Cl. A61B 3/00

(52) U.S. Cl. CPC A61B 3/0033

(57) ABSTRACT

An operating element for ophthalmological devices, in the form of a joystick in which existing, designated functions can be blocked and/or enabled in a targeted manner. The operating unit is in the form of a joystick and is provided for moving and/or controlling ophthalmological devices. According to the invention, at least one element is present for blocking a designated function and is designed such that the blocked function is enabled by removal of the element. For this purpose, the operating unit, designed as a joystick, has at the upper end an actuation unit with a press and/or rotate function. Although the present operating element is intended for ophthalmological devices, it can in principle also be used for joysticks that are employed in other technical fields, in order to change their functionality.

### CLAIM TO PRIORITY

This application claims priority to German Patent Application DE 10 2023 204 251.7 filed May 9, 2023 entitled “Methode zur kontrollierten Freigabe von Funktionalitäten eines Bedienelements.”

### TECHNICAL FIELD

The present invention relates to an operating element for ophthalmological devices, in the form of a joystick in which existing, designated functions can be selectively blocked and/or enabled in a targeted manner.

### BACKGROUND

Operating elements in the form of a joystick have also become established for operating, i.e. for moving and/or controlling, ophthalmological devices. The joysticks are in these cases provided for example for moving the main unit of the ophthalmological device relative to the base of the device in two axes and for aligning it relative to the patient's eye. For example, the joysticks also have control buttons and/or release switches for controlling various functions of the ophthalmological device.

Accordingly, many such arrangements are known from the prior art.

A first group of joysticks are designed primarily for moving and positioning ophthalmological devices. In this regard, reference is made by way of example to the following documents: DE 10 2006 015 051 A1, US 2009/0079939 A1 and US 2014/0368791 A1.

A second group of joysticks are designed more for operating and controlling the ophthalmological devices. For this purpose, the joysticks have an increased functionality, so that they are suitable not only for moving and positioning, but also for operating and controlling the ophthalmological devices. In this regard, reference is made by way of example to the following documents:

U.S. Pat. No. 5,532,769 A, DE 102 54 369 A1, US 2012/0050670 A1 and WO 2021/058092 A1.

A particular disadvantage of the known solutions is that it is possible only to a limited extent to retrofit additional functions on an existing joystick, i.e. a joystick that is already in use. This would therefore require customers, when ordering a device, to already know exactly in which configuration they want to operate the device. If the customer later wishes to change or extend the joystick, this involves considerable effort and costs, since the mechanical and/or electrical modification of a joystick cannot normally be carried out by the customer, but requires the input of a service employee.

### SUMMARY

Example embodiments of the invention make available an operating element for ophthalmological devices, in the form of a joystick in which existing, designated functions can be blocked and/or enabled in a targeted manner. For example, provision is made that the targeted enabling of functions can also be carried out by the users themselves.

In the operating unit for ophthalmological devices according to an example embodiment, with targeted blocking and/or enabling of individual functions, at least one element is present for blocking a designated function and is designed such that the blocked function is enabled by removal of the element.

According to a first example embodiment, the operating unit is designed as a joystick and at the upper end has an actuation unit with a press and/or rotate function. In this case, the actuation unit includes an actuation element and a holding element, which are both connected to the joystick via a connecting element, wherein the connection of the actuation element is movable and that of the holding element is rigid.

According to a second example embodiment, its surface is designed in accordance with the function. For example, for a press function, its surface is smooth or has a rotationally symmetrical structure or depression.

By contrast, the actuation element with a rotate function is designed such that its surface has a radially extending structure and/or its edge has a fluted structure.

Further embodiments relate to exemplary embodiments for operating units with a press function and/or rotate function which are blocked by application of an element for blocking a designated function and are enabled, individually or jointly, by removal of the element.

Although the present operating element is intended in particular for ophthalmological devices, it can in principle also be used for joysticks that are employed in other technical fields, in order to change their functionality.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 depicts possible embodiments of actuation units with a press and/or rotate function;

FIG. 2 depicts an operating unit designed as a joystick, having an actuation unit with a (blocked) rotate function;

FIG. 3 depicts an operating unit designed as a joystick, having an actuation unit with a press function and (blocked) rotate function;

FIG. 4 depicts an operating unit designed as a joystick, having an actuation unit with a (blocked) press function and a (blocked) rotate function; and

FIG. 5 depicts an operating unit designed as a joystick, having an actuation unit with a (blocked) press function and a (separately blocked) rotate function.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

The operating unit for ophthalmological devices is configured in the form of a joystick and is provided for moving and/or controlling ophthalmological devices.

According to example embodiments the invention, at least one element is present for blocking a designated function and is designed such that the blocked function is enabled by removal of the element. For this purpose, the operating unit, configured as a joystick, has at the upper end an actuation unit with a press and/or rotate function.

The actuation unit includes an actuation element and a holding element, which are both connected to the joystick via a connecting element, wherein the connection of the actuation element is movable and that of the holding element is rigid.

An actuation element having a press function is configured for example in such a way that the surface of the actuation element is smooth or has a rotationally symmetrical structure.

An actuation element having a rotate function is configured for example in such a way that the surface of the actuation element has a radially extending structure and/or its edge is fluted.

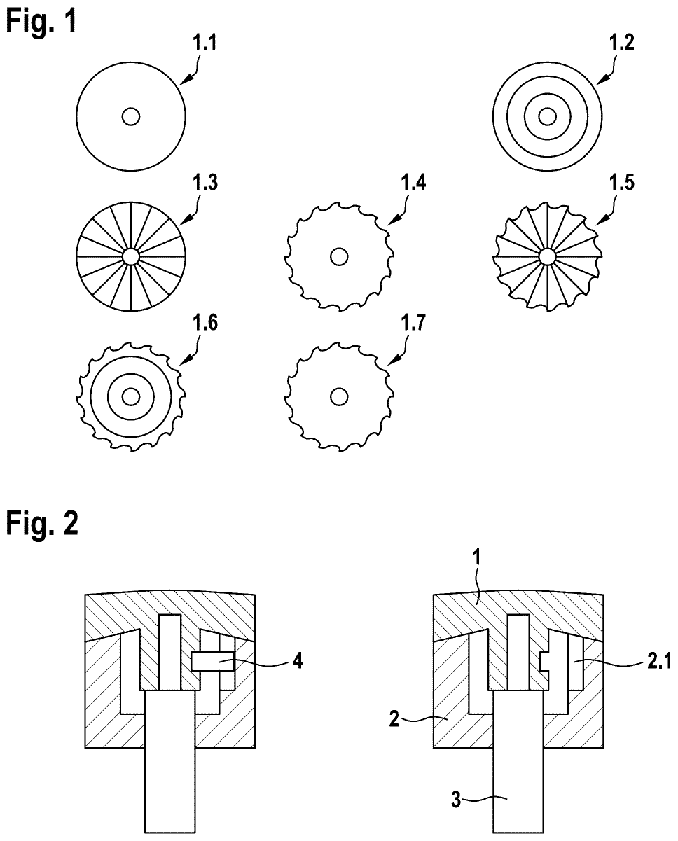

In this regard, FIG. 1 shows possible embodiments of actuation units with a press and/or rotate function, each in a plan view.

The upper illustrations show actuation elements with a press function, which have a smooth surface 1.1 or a rotationally symmetrically structured surface 1.2.

The central illustrations show actuation elements with a rotate function, which have a surface with a radially extending structure 1.3, with a fluted edge 1.4 or with both 1.5.

The lower illustrations show actuation elements with a press and rotate function, which have a rotationally symmetrically structured surface and a fluted edge 1.6 or have a smooth surface and a fluted edge 1.7.

According to a first example embodiment variant, the actuation element is designed such that it rests lightly on the holding element and thus, whilst a press function is unavailable, a rotate function is available, which can be blocked by an element arranged between the actuation element and a holding element.

For this purpose, the actuation element has an opening for receiving, with a precise fit, the element for blocking the rotate function, while a vertical groove is formed in the holding element, into which vertical groove the element for blocking the rotate function engages, and, by removal of the element for blocking a designated function, only the blocked rotate function is enabled.

In this regard, FIG. 2 shows an operating unit designed as a joystick having an actuation unit with a (blocked) rotate function.

The actuation unit consists of an actuation element 1 and a holding element 2, which are both connected to the joystick (not shown) via a connecting element 3, wherein the actuation element 1 rests lightly on the holding element 2. An element 4 for blocking the rotate function is arranged between the actuation element 1 and the holding element 2.

For this purpose, the actuation element 1 has an opening for receiving, with a precise fit, the element 4 for blocking the rotate function. The holding element 2 has a vertical groove 2.1 into which the element 4 for blocking the rotate function engages. By removal of the element 4 for blocking the rotate function, the latter is enabled.

According to a second example embodiment variant, the actuation element is configured such that it is connected to the holding element with a spacing, such that both a press function and a rotate function are available, wherein the rotate function can be blocked by an element arranged between the actuation element and a holding element.

For this purpose, the actuation element has an opening for receiving, with a precise fit, the element for blocking the rotate function, while a vertical groove is formed in the holding element, into which vertical groove the element for blocking the rotate function engages. The length of the groove is dimensioned such that a press function remains available and only the rotate function is blocked.

By removal of the element for blocking the blocked rotate function, the blocked rotate function is enabled.

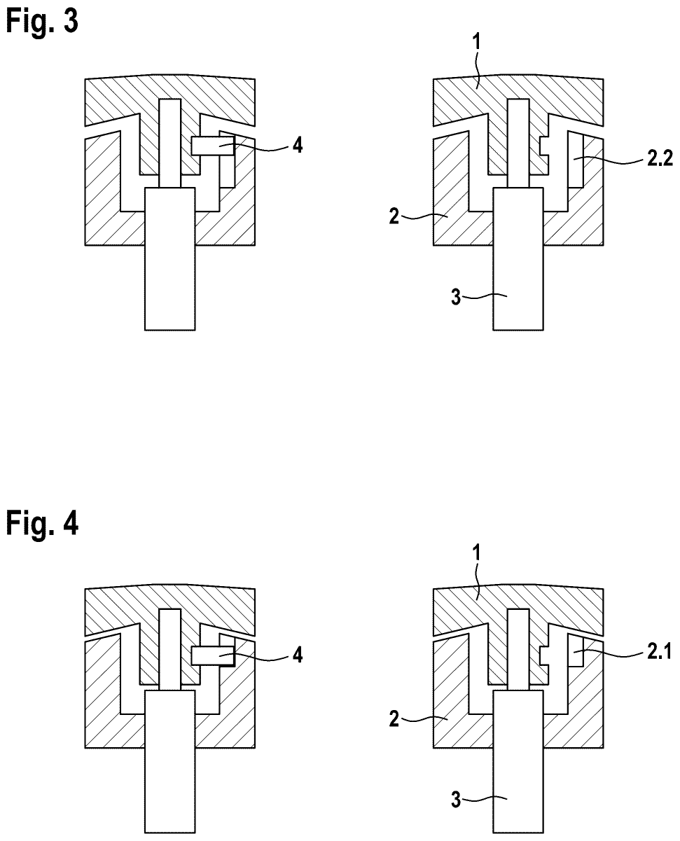

In this regard, FIG. 3 depicts an operating unit designed as a joystick having an actuation unit with a press function and (blocked) rotate function.

The actuation unit includes an actuation element 1 and a holding element 2, which are both connected to the joystick (not shown) via a connecting element 3, wherein the actuation element 1 is connected to the holding element 2 with a spacing. An element 4 for blocking the rotate function is arranged between the actuation element 1 and the holding element 2.

For this purpose, the actuation element 1 has an opening for receiving, with a precise fit, the element 4 for blocking the rotate function. The holding element 2 has a vertical groove 2.2, the length of which is dimensioned such that a press function remains available and only a rotate function is blocked. By removal of the element 4 for blocking the rotate function, the latter is also enabled.

According to a third example embodiment variant, the actuation element is designed such that it is connected to the holding element with a spacing, such that a press function and a rotate function are available wherein an element for blocking the designated functions is arranged between the actuation element and a holding element.

For this purpose, the actuation element has an opening for receiving, with a precise fit, the element for blocking the designated functions, while a vertical groove is formed in the holding element, into which vertical groove the element for blocking a designated function engages, wherein the length of the groove is dimensioned such that neither a press function nor a rotate function is available.

By removal of the element for blocking the designated function, both the blocked press function and the blocked rotate function are enabled.

In this regard, FIG. 4 shows an operating unit designed as a joystick having an actuation unit with a (blocked) press function and (blocked) rotate function.

The actuation unit consists of an actuation element 1 and a holding element 2, which are both connected to the joystick (not shown) via a connecting element 3, wherein the actuation element 1 is connected to the holding element 2 with a spacing. An element 4 for blocking the press and rotate function is arranged between the actuation element 1 and the holding element 2.

For this purpose, the actuation element 1 has an opening for receiving, with a precise fit, the element 4 for blocking the designated functions. The holding element 2 has a vertical groove 2.1, the length of which is dimensioned such that both the press function and the rotate function are blocked. By removal of the element 4 for blocking the designated functions, both the press function and the rotate function are enabled.

According to a fourth embodiment variant, the actuation element is designed such that it is connected to the holding element with a spacing, such that a press function and a rotate function are available. In this embodiment variant, two elements, each for blocking a respective designated function, are arranged between the actuation element and the holding element.

For this purpose, the actuation element has an opening for receiving, with a precise fit, the first element for blocking the rotate function, while a vertical groove is formed in the holding element, into which vertical groove the first element for blocking the rotate function engages. A second element for blocking the press function is designed as a spacer for closing the distance between the actuation element and the holding element.

By removal of one or both of the elements for blocking a designated function, the blocked press function and/or the blocked rotate function can be enabled.

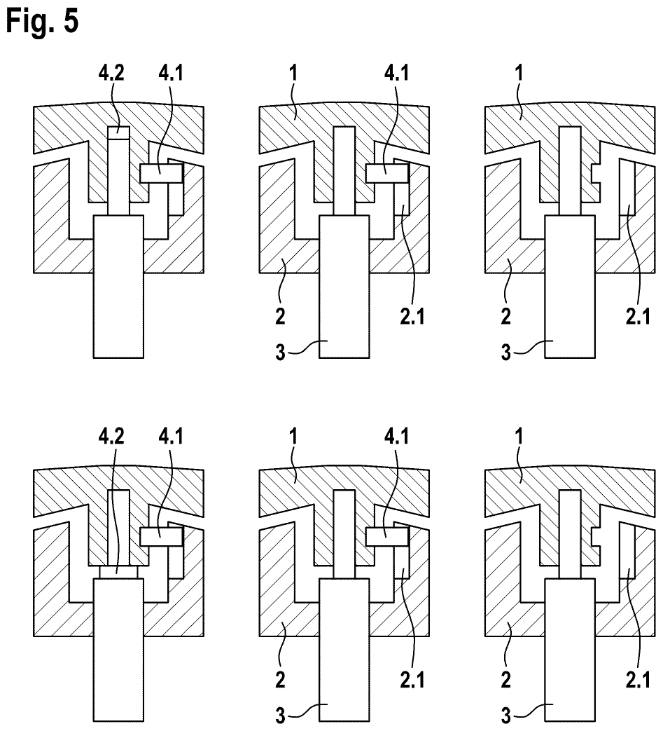

In this regard, FIG. 5 shows two variants of an operating unit designed as a joystick, having an actuation unit with a (blocked) press function and (separately blocked) rotate function.

The actuation unit 1 consists of an actuation element 1 and a holding element 2, which are both connected to the joystick (not shown) via a connecting element 3, wherein the actuation element 1 is connected to the holding element 2 with a spacing. A first element 4.1 for blocking the rotate function and a second element 4.2 for blocking the press function are arranged between the actuation element 1 and the holding element 2.

For this purpose, the actuation element 1 has an opening for receiving, with a precise fit, the element 4.1 for blocking the rotate function. The holding element 2 has a vertical groove 2.1, the length of which is dimensioned such that the press function would be possible if the second element 4.2 for blocking the press function were removed.

By removal of the second element 4.2 for blocking the press function, only the press function would be enabled (illustration in the middle); for enabling the rotate function, the element 4.1 for blocking the rotate function must be removed (illustration on the right).

With the arrangement according to the invention, an operating element for ophthalmological devices is made available in the form of a joystick, in which existing, designated functions can be blocked and/or enabled in a targeted manner.

In particular, it is possible that the targeted enabling of functions can also be carried out by the users themselves. In addition, the invention permits step by step “enabling” of the various functionalities, which was not hitherto available.

With a fully mechanically and electronically tested operating element, the solution according to the invention offers the possibility of permitting four different configurations of two functionalities simply by replacing a single part.

Assuming that the two functionalities are a press function (e.g. to trigger an image capture or to start/stop a film/video sequence) and a rotate function (e.g. to increase/decrease the exposure time or aperture or camera sensitivity or image cropping, etc.), a large number of configurations are possible with the operating elements of the invention.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112 (f) are not to be invoked unless the specific terms “means for” or “step for” are recited in a claim.

1. An operating unit for use with ophthalmological devices, comprising
targeted blocking of individual functions, targeted enabling of individual functions or both; and
at least one element configured to block a designated function and further configured such that the designated blocked function is enabled by removal of the element.

2. The operating unit according to claim 1, wherein the operating unit is configured as a joystick; and
the joystick comprises at an upper end thereof an actuator with a press function, a rotate function or both.

3. The operating unit according to claim 1, wherein the actuator comprises an actuation element and a holding element, which are both connected to the joystick via a connecting element, wherein a first connection of the actuation element is movable and a second connection of the holding element is rigid.

4. The operating unit according to claim 1, wherein the actuation element has a press function and is configured such that a surface of the actuation element is smooth or presents a rotationally symmetrical structure or depression.

5. The operating unit according to claim 1, wherein the actuation element has a rotate function and is configured such that a surface of the actuation element has a radially extending structure, an edge of the actuation element is fluted or both.

6. The operating unit according to claim 1, wherein the actuation element rests lightly on the holding element, so that a rotate function but no press function is available, wherein an element that blocks a designated function is arranged between the actuation element and a holding element, wherein the actuation element has an opening for receiving, with a precise fit, the element that blocks the rotate function, while a vertical groove is formed in the holding element, wherein the element that blocks the rotate function engages into the vertical groove, and wherein removal of the element that blocks the designated function, only the blocked rotate function is enabled.

7. The operating unit according to claim 1, wherein the actuation element is connected to the holding element with a spacing, such that a press function and a rotate function are available, wherein an element that blocks the designated function is arranged between the actuation element and a holding element, in that the actuation element has an opening to receive, with a precise fit, the element that blocks the rotate function, while a vertical groove is formed in the holding element, wherein the element that blocks the rotate function engages into which vertical groove, wherein the length of the groove is dimensioned such that a press function remains available and wherein removal of the element that blocks a designated function enables the blocked rotate function.

8. The operating unit according to claim 1, wherein the actuation element is connected to the holding element with a spacing, such that a press function and a rotate function are available, wherein an element that blocks the designated function is arranged between the actuation element and a holding element, wherein the actuation element has an opening for receiving, with a precise fit, the element that blocks a designated function, while a vertical groove is formed in the holding element, wherein the element that blocks the designated function engages in the vertical groove, wherein the length of the groove is dimensioned such that neither a press function nor a rotate function is available and wherein removal of the element that blocks the designated function, enables the blocked press function and the blocked rotate function.

9. The operating unit according to claim 1, wherein the actuation element is connected to the holding element with a spacing, such that a press function and a rotate function are available, wherein two elements, each that block a respective designated function, are arranged between the actuation element and the holding element,
wherein the actuation element has an opening for receiving, with a precise fit, a first element that blocks the rotate function, while a vertical groove is formed in the holding element, wherein the first element that blocks the rotate function engages in the vertical groove, wherein a second element that blocks the press function is configured as a spacer in order to close a distance between the actuation element and the holding element, and wherein, by removal of one or both of the elements that block the designated function, the blocked press function, the blocked rotate function or both are enabled.
